(19) United States
(12) Patent Application Publication
(10) Pub. No.: US 2013/0005747 A1
(43) Pub. Date: Jan. 3, 2013

(54) METHOD FOR SELECTING A CANCER THERAPY

(75) Inventors: Simon Richard GREEN, Cardiff (GB); Judy H. CHIAO, Berkeley Heights, NJ (US); Jeffery SETTLEMAN, Mill Valley, CA (US); Ethan DMITROVSKY, Hanover, NH (US); Fabrizio Vittorio GALIMBERTI, Cleveland Heights, OH (US)

(73) Assignees: Cyclacel Limited, London (GB); The General Hospital Corporation d/b/a Massachusetts General Hospital, Boston, MA (US); The Trustees of Dartmouth College, Hanover, NH (US)

(21) Appl. No.: 13/333,219

(22) Filed: Dec. 21, 2011

Related U.S. Application Data
(60) Provisional application No. 61/425,621, filed on Dec. 21, 2010.

(51) Int. Cl. C12Q 1/68; A61P 35/00; A61K 31/52

(52) U.S. Cl. 514/263.4; 435/6.11

(57) ABSTRACT

The present invention provides a method for determining whether or not a cancer subject is suitable for treatment with a purine-based roscovitine-like inhibitor, which method comprises the step of determining the ras status of the cancer, wherein a determination that the subject has mutant ras status is indicative that the subject is suitable for treatment with a purine-based roscovitine-like inhibitor.

### RELATED APPLICATIONS

The present application claims priority to U.S. Provisional Application No. 61/425,621, filed Dec. 21, 2010. The entire contents of this application are hereby incorporated herein by reference in their entirety.

### GOVERNMENT FUNDING

This invention was made with government support under contract numbers CA087546 and CA111422 awarded by the National Institutes of Health. The government has certain rights in the invention.

### FIELD OF THE INVENTION

The present invention relates to a method for determining which cancer subject is suitable for treatment with a particular class of inhibitors, to a method for selecting a therapy for a cancer subject and to a method of treatment using such therapy.

### BACKGROUND TO THE INVENTION

Roscovitine is a cyclin dependent kinase (CDK) inhibitor which selectively inhibits multiple enzyme targets, such as CDK2/E, CDK2/A, CDK7 and CDK9, that are central to the process of cell division and cell cycle control. Preclinical studies have shown that roscovitine works by inducing cell apoptosis, or cell suicide, in multiple phases of the cell cycle.

Roscovitine has been shown to have anti-tumor activity against many human cancer cell lines, including those of breast, prostate, colorectal, pancreatic and lung cancer origins (Fleming et al (2008) Clin. Cancer Res. 14:4326-35).

Roscovitine has also been evaluated in several Phase I and II studies (in approximately 400 patients) and has shown early signs of anti-cancer activity. Studies include a Phase I study in which single agent roscovitine was administered to patients with advanced cancer including NSCLC and two Phase IIa studies in which roscovitine was administered in combination with gemcitabine and cisplatin as first-line treatment and with docetaxel as second-line treatment in NSCLC. Roscovitine has also been evaluated in a Phase I study in patients with nasopharyngeal cancer (NPC) with evidence of tumor shrinkage and concomitant reduction in copy counts of the EBV virus that is causally associated with the pathogenesis of NPC.

Roscovitine is currently being evaluated in the APPRAISE trial, a Phase 2b randomized double-blinded study to evaluate the efficacy and safety of the drug as a third line or later treatment in patients with NSCLC. The trial is using a randomized discontinuation trial design. Roscovitine is also being evaluated in a Phase 2 study as a single agent in patients with nasopharyngeal cancer.

Although roscovitine and purine-based roscovitine-like inhibitors have been shown to be effective against a range of cancers, efficacy varies between cancer types and between individual cancer-patients.

It is desirable, therefore, for an improved method to predict the likely efficacy for this type of treatment for a given cancer patient prior to commencing treatment.

### DESCRIPTION OF THE FIGURES

FIG. 1—Chemical structure of R-roscovitine (also known as CYC202 and as seliciclib)

FIG. 2—Chemical structures of Compounds A, B, C and D

FIG. 3—Chemical structure for Bohemine

FIG. 4—Chemical structure for Olomoucine

FIG. 5—Profiling for roscovitine sensitivity revealed growth inhibitory effects in diverse cancer lines. (A) Schematic representation of roscovitine sensitivity across 270 cancer cell lines from diverse tissues. Lung (others), four small-cell lung cancer, six mesothelioma, and one bronchial carcinoma cell lines. Miscellaneous, two fibrosarcoma, one fibrous histiocytoma, and one small round-cell sarcoma cancer cell lines. The complete set of data is presented in Table 1. (B) Pie chart representation of NSCLC cell lines sensitivity to roscovitine treatment. (C) Left, ras status for the 15 NSCLC cell lines with highest growth inhibitor response to roscovitine. Right, ras status for the 15 NSCLC cell lines with least growth inhibitory response to roscovitine.

FIG. 6—Effects of seliciclib treatment on human and murine lung cancer cell lines versus murine immortalized pulmonary epithelial cells and cooperation with taxanes. A) Seliciclib treatment independently caused dose-dependent growth inhibition of H-23 (left panel), HOP-62 (center panel) and H-522 (right panel) human lung cancer cell lines at 72 hours post-treatments. B) Dose-dependent growth inhibition of C-10 murine immortalized pulmonary epithelial cells following seliciclib treatment or vehicle treatment. C) Combined treatment of seliciclib with paclitaxel or docetaxel cooperatively inhibited proliferation of these human lung cancer cell lines.

### SUMMARY OF THE INVENTION

The present inventors have surprisingly found that, within cancer cell types, there is a tight correlation between ras status and the sensitivity of the cell to treatment with purine-based roscovitine-like inhibitors. Activating ras mutations are associated with increased sensitivity to this type of treatment.

This finding is particularly surprising given that, in general, patients that express a mutant ras protein appear to be less responsive than their wild type equivalents to a wide range of chemotherapies including both traditional cytotoxics (e.g. irinotecan) and more novel targeted agents (e.g. EGFR inhibitors and mTOR inhibitors).

The finding enables predictions to be made about how likely a given cancer patient is to respond to and benefit from treatment with a purine-based roscovitine-like inhibitor.

Thus in a first aspect, the present invention provides a method for determining whether or not a cancer subject is suitable for treatment with a purine-based roscovitine-like inhibitor, which method comprises the step of determining the ras status of the cancer, wherein a determination that the subject has mutant ras status is indicative that the subject is suitable for treatment with a purine-based roscovitine-like inhibitor.

The present inventors also found that there is a tight correlation between the absence of a ras mutation and reduced sensitivity to roscovitine treatment. Hence, in the method of the invention a determination that the subject has wild-type ras status is indicative that the subject is unsuitable for treatment with a purine-based roscovitine-like inhibitor.

In a second aspect, the present invention provides a method for selecting a therapy for treating a cancer subject which comprises the step of determining whether the subject is suitable for treatment with a purine-based roscovitine-like inhibitor using a method according to the first aspect of the invention, and selecting treatment with a purine-based roscovitine-like inhibitor if the subject has mutant ras status.

If, using the method of the second aspect of the invention, the subject is determined to have wild-type ras status, then this is a negative indicator that treatment with a purine-based roscovitine-like inhibitor will be effective. This may form part of a decision to select an alternative type of treatment. However, if there are other positive indicators which would support treatment with a purine-based roscovitine-like inhibitor then the fact that the subject has wild-type ras may not rule out this type of treatment.

The treatment may involve the use of a purine-based roscovitine-like inhibitor in combination with another therapeutic agent.

The treatment may involve the use of a purine-based roscovitine-like inhibitor in combination with a receptor tyrosine kinase (RTK) inhibitor.

The treatment may involve the use of a purine-based roscovitine-like inhibitor in combination with an EGF-R inhibitor.

The treatment may involve the use of a purine-based roscovitine-like inhibitor in combination with an m-TOR inhibitor.

The treatment may involve the use of a purine-based roscovitine-like inhibitor in combination with a PI3-kinase inhibitor.

The treatment may involve the use of a purine-based roscovitine-like inhibitor in combination with a MEK inhibitor

The treatment may involve the use of a purine-based roscovitine-like inhibitor in combination with a prodrug or pharmaceutical preparation in which the active ingredient is a microtubule targeting agent.

The microtubule targeting agent may, for example, be paclitaxel, docetaxel or taxane.

The purine-based roscovitine-like inhibitor may be, for example, roscovitine, Compounds A B, C, D and E, bohemine or olomoucine.

The subject having mutant ras status may express K-ras, H-ras or N-ras mutant protein.

The cancer may be selected from, for example lung, pancreas, colorectal, breast, liver, intestine, oesophagus, uterus, skin, head & neck, nasopharyngeal and haematological cancer, such as Acute Myeloid Leukemia (AML).

In particular, the cancer may be lung or colorectal cancer. The cancer may be non small-cell lung carcinoma (NSCLC).

The cancer may be insensitive to chemotherapy with other agents. For example, the cancer may be insensitive to chemotherapy with cytotoxic agents. The cancer may be insensitive to treatment with targeted agents such as EGFR inhibitors and mTOR inhibitors.

### DETAILED DESCRIPTION

### I. Purine Based Roscovitine-Like Inhibitor

The purine-based roscovitine-like inhibitor may be a purine substituted at position 2 by an aliphatic amine, and substituted at position 6 by a benzylic amine where the aromatic moiety may be either heteroaryl or aryl, and substituted at position 9 by an aliphatic group. In each case the 2, 6 and 9-position substituents may independently bear optional substituents.

The purine-based roscovitine-like inhibitor may be selected from the group consisting of: roscovitine, Compounds A B, C and D bohemine and olomoucine.

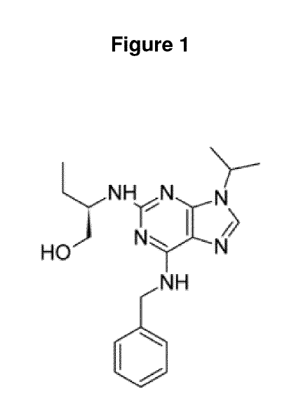

Roscovitine (seliciclib or CYC202) is a 2,6,9-trisubstituted purine analogue. The chemical structure of roscovitine is shown in FIG. 1.

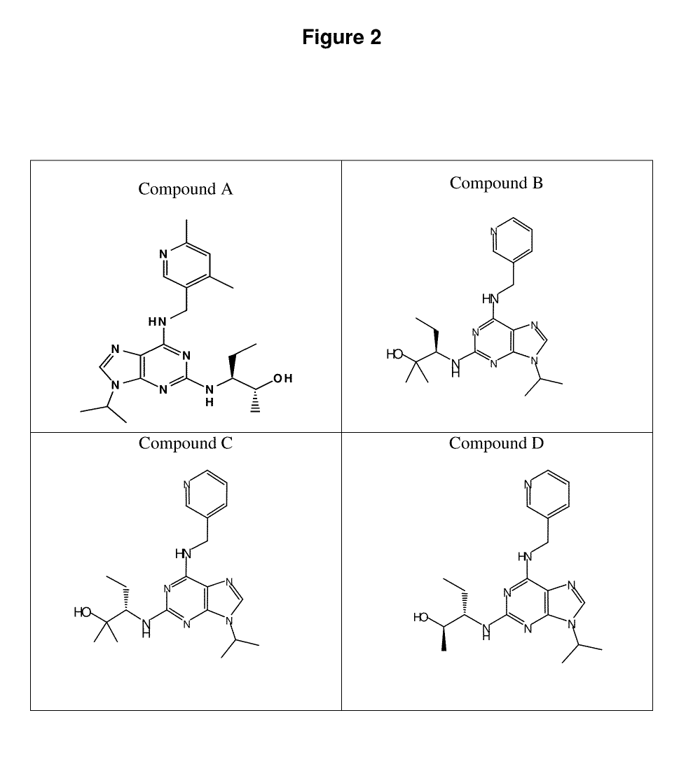

The chemical structures of compounds A, B, C and D are shown in FIG. 2.

Compound A has the chemical name (2R,3S-3-(6-((4,6-dimethylpyridin-3-ylmethylamino)-9-isopropyl-9H-purin-2-ylamino)pentan-2-ol.

Compound B has the chemical name (3R)-3-{9-isopropyl-6-[(pyridin-3-ylmethyl)-amino]-9H-purin-2-ylamino}-2-methyl-pentan-2-ol.

Compound C has the chemical name (3S)-3-{9-isopropyl-6-[(pyridin-3-ylmethyl)-amino]-9H-purin-2-ylamino}-2-methyl-pentan-2-ol.

Compound D has the chemical name (2R,3S)-3-({9-isopropyl-6-[(pyridin-3-yl methyl)amino]-9H-purin-2-yl}amino)pentan-2-ol

Compounds B, C and D are oral multikinase inhibitors with very similar CDK inhibitory profiles to roscovitine.

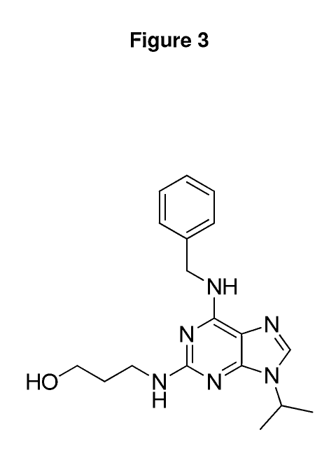

Bohemine having the chemical name having the chemical name 6-Benzylamino-2-(3-hydroxypropylamino)-9-isopropylpurine has the chemical structure shown in FIG. 3.

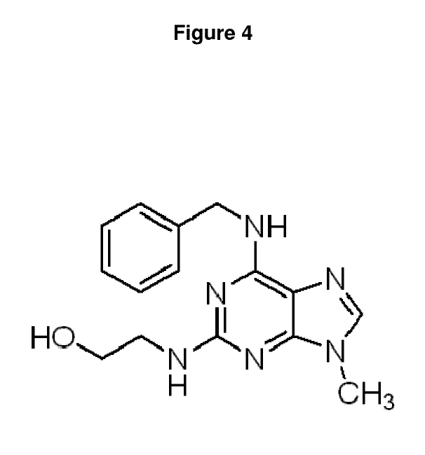

Olomoucine, having the chemical name 2-[[9-methyl-6-[(phenylmethyl)amino]-9H-purin-2-yl]amino]-ethanol has the chemical structure shown in FIG. 4.

The purine-based roscovitine-like inhibitor may be a purine of formula (I),

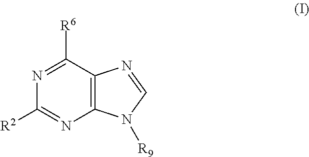

wherein:
R² is NR⁴R⁵, where R⁴ is H or alkyl, and R⁵ is alkyl, wherein each alkyl group is independently optionally substituted by one or more R¹ substituents; preferably, R⁴ is H and R⁵ is alkyl optionally substituted by one or more OH groups;
R⁶ is NHR³, where R³ is aralkyl or alkyl-heteroaryl, each of which is optionally substituted by one or more R¹ substituents; preferably, R³ is —CH₂-phenyl or —CH₂-pyridinyl, wherein the phenyl or pyridinyl is optionally substituted;
R⁹ is alkyl, cycloalkyl or cycloalkyl-alkyl, each of which is optionally substituted by one or more R¹ substituents; preferably R⁹ is alkyl;
each R¹ is independently selected from alkyl, OR⁷, NR⁷R⁸, halogen, CF₃, NO₂, COR⁷, CN, COOR⁷, CONR⁷R⁸, SO₂R⁷ and SO₂NR⁷R⁸, where each R⁷ and R⁸ is independently H or alkyl;
or a pharmaceutically acceptable salt or ester thereof.

As used herein, the term “alkyl” includes both saturated straight chain and branched alkyl groups. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl.

As used herein, the term “cycloalkyl” refers to a cyclic alkyl group. Preferably, the cycloalkyl group is a C₃₋₁₂ cycloalkyl group.

As used herein, the term “cycloalkyl-alkyl” refers to a group having both cycloalkyl and alkyl functionalities.

As used herein, the term “aryl” refers to a C₆₋₁₂ aromatic group which may be substituted (mono- or poly-) or unsubstituted. Typical examples include phenyl and naphthyl etc. Suitable substituents include, for example, one or more R¹ groups.

As used herein, the term “heteroaryl” refers to a C₂₋₁₂ aromatic, substituted (mono- or poly-) or unsubstituted group, which comprises one or more heteroatoms. Preferably, the heteroaryl group is a C₄₋₁₂ aromatic group comprising one or more heteroatoms selected from N, O and S. Suitable heteroaryl groups include pyrrole, pyrazole, pyrimidine, pyrazine, pyridine, quinoline, thiophene, 1,2,3-triazole, 1,2,4-triazole, thiazole, oxazole, iso-thiazole, iso-oxazole, imidazole, furan and the like. Again, suitable substituents include, for example, one or more R¹ groups.

As used herein, the term “aralkyl” includes, but is not limited to, a group having both aryl and alkyl functionalities. By way of example, the term includes groups in which one of the hydrogen atoms of the alkyl group is replaced by an aryl group, e.g. a phenyl group optionally having one or more substituents such as halo, alkyl, alkoxy, hydroxy, and the like. Typical aralkyl groups include benzyl, phenethyl and the like.

As used herein the term “alkyl-heteroaryl” includes, but is not limited to, a group having both heteroaryl and alkyl functionalities as described above.

The invention also encompasses all enantiomers and tautomers of the purine-based roscovitine-like inhibitors. For compounds that posses optical properties (one or more chiral carbon atoms) or tautomeric characteristics, the corresponding enantiomers and/or tautomers may be isolated/prepared by methods known in the art.

### II. Determination of Ras Status

The ras genes were first identified as the transforming oncogenes, responsible for the cancer-causing activities of the Harvey (the HRAS oncogene) and Kirsten (KRAS) sarcoma viruses. Subsequent studies identified a third human ras gene, designated NRAS, for its initial identification in human neuroblastoma cells.

The three human ras genes encode highly related 188 to 189 amino acid proteins, designated H-Ras, N-Ras and K-Ras4A and K-Ras4B (the two K-Ras proteins arise from alternative gene splicing).

Mutations in the Ras family of proto-oncogenes (comprising H-Ras, N-Ras and K-Ras) are very common, being found in 20% to 30% of all human tumors.

Ras proteins are GTP-coupled proteins that are important in receptor tyrosine kinase signalling. Mutations in the Ras protein usually cause constitutive activation of Ras GTPase which leads to overactivation of downstream signalling pathways, resulting in cell transformation and tumorigenesis.

Ras status may be determined by a variety of methods known in the art including quantitative PCR (Q-PCR) using mutation specific primers in kits such as the DxS TheraScreen K-RAS Kit or standard PCR-restriction fragment length polymorphism methodology as has been reported previously (Hatzaki et al., Molecular and Cellular Probes 2001; v15, 243) or and direct sequencing using standard techniques of DNA samples isolated from patient tumor biopsies. Any method which gives a high level of accuracy and precision is suitable for use in connection with the method of the invention.

In particular, RAS status may be determined by a method which involves the following steps:

i) Sequence analysis of the H-Ras, K-Ras and/or N-ras genes
ii) Comparison of the sequence with the wild-type H-Ras, K-Ras and/or N-ras genes to determine whether there is an activating ras mutation.

KRAS mutational analysis is commercially available from a number of laboratories such as Clarient Inc or Quintiles.

Two anti-EGFR monoclonal antibody drugs (panitumumab (Vectibix) and cetuximab (Erbitux)) are indicated for treatment of metastatic colorectal cancer. Vectibix specifies that it is are only suitable for treatment of subjects not having KRAS mutations i.e. the patients must be RAS wild type (http://pi.amgen.com/united_states/vectibix/vectibix_pi.pdf).

### III. Treatment

The treatment may involve the use of a purine-based roscovitine-like inhibitor in combination with another therapeutic agent.

The two or more agents may be administered in combination, separately or sequentially.

The agent may be a prodrug in which one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. An example of such a modification is an ester, wherein the reversion may be carried out by an esterase.

The purine-like roscovitive inhibitor, other agent or combination may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, use may be made of compressed tablets, pills, tablets, gellules, drops, and capsules. The compositions may contain from 1 to 2000 mg, for example, from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The purine-like roscovitive inhibitor, other agent or combination may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

The purine-like roscovitive inhibitor, other agent or combination may be administered intravenously.

A person of ordinary skill in the art can easily determine an appropriate dose of the purine-like roscovitive inhibitor, other agent or combination to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.1 to 30 mg/kg body weight, such as from 0.1 to 10 mg/kg, more preferably from 2 to 20 mg/kg body weight.

Roscovitine is typically administered from about 0.05 to about 5 g/day, preferably from about 0.4 to about 3.2 g/day. Roscovitine is preferably administered orally in tablets or capsules. The total daily dose of roscovitine can be administered as a single dose or divided into separate dosages administered two, three or four times a day.

### IV. Subject

The subject may be a mammalian subject, such as human subject.

The present invention provides a method for determining whether a cancer subject is suitable for treatment with a purine-based roscovitine-like inhibitor. A cancer subject is “suitable” if they are likely to respond to treatment with a purine-based roscovitine-like inhibitor or likely to benefit from purine-based roscovitine-like inhibitor. A subject is “likely” to respond/benefit if they have a 20%, 30%, 40%, 50%, 60%, 70%, 80% or 90% chance of responding to treatment.

The subject may be a cancer patient, i.e. a subject having an existing cancerous condition. The subject may, for example, have one of the following cancers: cancer of the breast, ovary, cervix, prostate, testis, oesophagus, stomach, skin, lung, bone, colon, pancreas, thyroid, biliary passages, buccal cavity and pharynx, lip, tongue mouth, small intestine, colon-rectum, large intestine, rectum, brain and central nervous system, glioblastoma, neuroblastoma, keratocanthoma, epidermoid carcinoma, large cell carcinoma, adenocarcinoma, adenoma, follicular cancinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's disease and leukemia.

In particular, the cancer may be selected from lung, pancreas, colorectal, breast, liver, intestine, oesophagus, uterus, skin, head & neck, nasopharyngeal and haematological malignancies, such as acute myeloid leukemia (AML).

The cancer may be lung or colorectal cancer.

In particular the cancer may be non-small cell lung cancer (NSCLC).

The cancer may be of a type that is insensitive to other drug types, such as EGFR-inhibitors. The term “insensitive” indicates that following treatment, tumor growth has progressed (>20% increase) as defined by the Response Evaluation Criteria in Solid Tumours (RECIST) Committee.

The patient may have relapsed following treatment with another drug type. The term “relapsed” as used herein means that after initial improvement, the symptoms of cancer, such as rate of proliferation of cancer cells, returned. A patient may be considered to have relapsed when after a period of remission or stable disease, following treatment, the tumor has started to grow again (>20% increase above the smallest size since the start of treatment) as defined by the Response Evaluation Criteria in Solid Tumours (RECIST) Committee.

The invention will now be further described by way of Examples, which are meant to serve to assist one of ordinary skill in the art in carrying out the invention and are not intended in any way to limit the scope of the invention.

### EXAMPLES

### Example 1

### Pharmacogenomic Analysis of Cancer Cell Lines

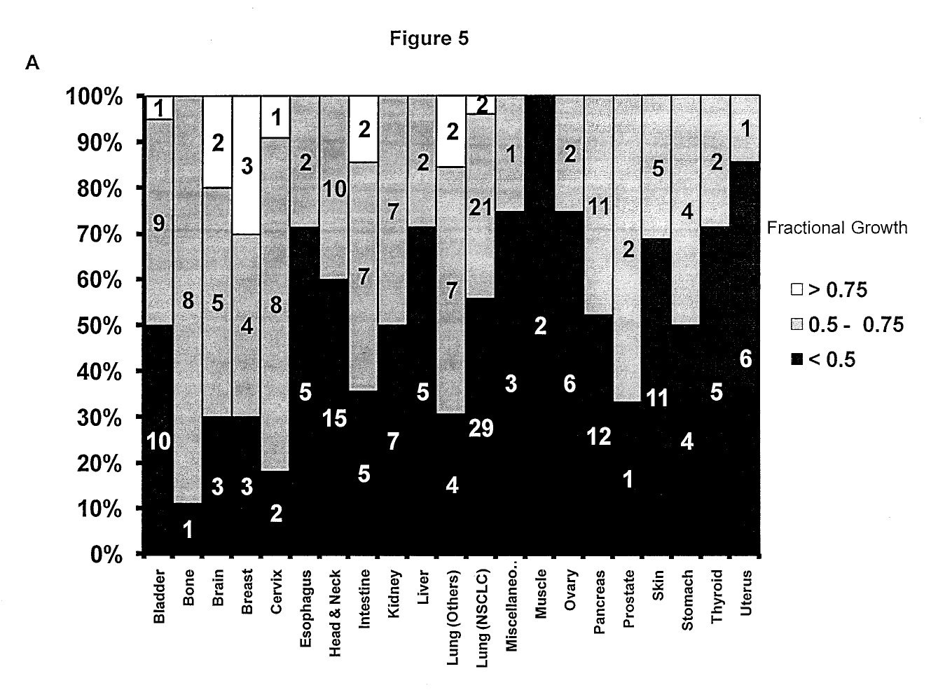

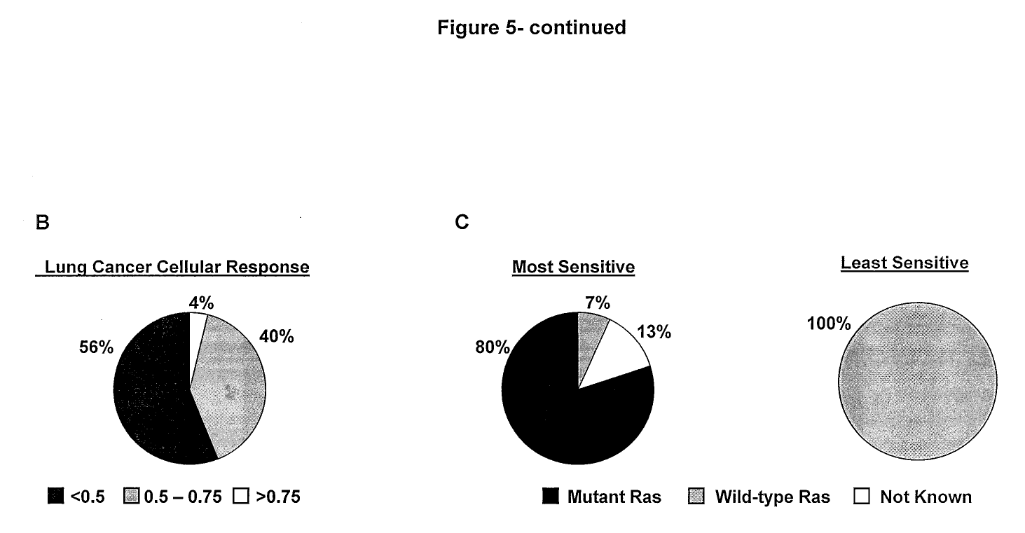

To examine the effects of roscovitine comprehensively, a method for detecting pharmacologic responses was used with a large number of cancer cell lines and a robotic-based platform (McDermott et al (2007) 104:19936-41; McDermott et al (2008) Methods Enzymol. 438:331-41). A total of 270 human cancer cell lines from diverse cancer histopathologic types were investigated. Over half of investigated lung, pancreatic, head and neck, esophageal, liver, thyroid, ovarian, uterine, and skin cancer cell lines showed at least 50% growth inhibition following 72 hours of roscovitine treatment, compared with vehicle treated cells (see Table 1; FIG. 5A). Among the 270 human cancer cell lines investigated, 52 were of NSCLC origin and 2 (4%) were relatively insensitive to roscovitine (fractional growth, ≧75% compared with vehicle-treated cells), whereas 21 (40%) displayed a modest sensitivity (fractional growth was between 75% and 50% compared with vehicle-treated cells), and 29 (56%) showed marked sensitivity scored as fractional growth, ≦50% versus controls (FIG. 5B).

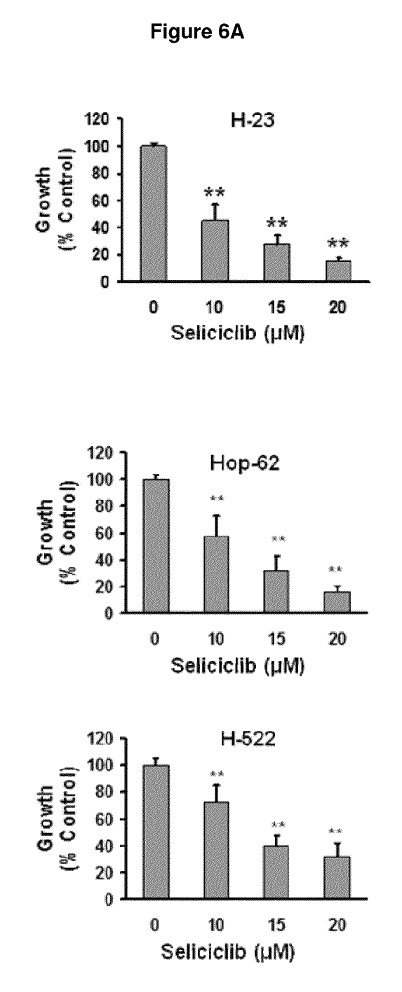

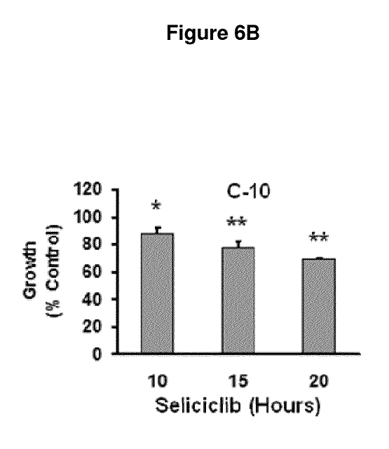

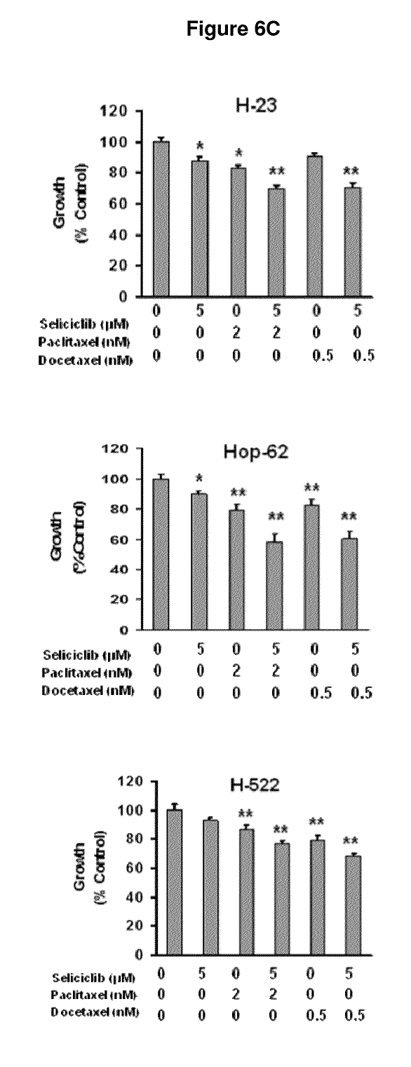

Effects of roscovitine treatments on proliferation of H522 lung cancer cells were also investigated (FIG. 6A) with concordant results as in this high-throughput experiment. As shown, this cell line was less sensitive than others examined and had wild-type ras status (Table 2). The ras status of 13 to 15 NSCLC cell lines with highest sensitivity to roscovitine is known. Intriguingly, analyses revealed that 12 of 13 (92%) of the lung cancer cells most sensitive to roscovitine treatment have K-ras- or N-ras-activating mutations, whereas none of the NSCLC cell lines with the least sensitivity to roscovitine had such mutations (Table 2; FIG. 5C). Prior work has shown that both H23 and HOP-62 lung cancer cells harbor ras mutations and, as shown in Table 1, are more sensitive to roscovitine that H-522 lung cancer cells. Thus, findings from this large panel of cancer cell lines indicated significant roscovitine sensitivity well beyond those human and murine lung cancer cells already investigated. For lung cancer cells, this response is tightly associated with the presence of ras activation in highly responsive cells.

A tight correlation was therefore found between ras mutations and sensitivity to roscovitine treatment. Activating ras mutations are found in a subset of NSCLCs and this predicts resistance to epidermal growth factor receptor-tyrosine kinase inhibitors (Massarelli et al (2007) 13:2890-2896; Eberhard et al (2005) J. Clin. Oncol. 23:5900-9). The presence of ras mutations has been linked to chromosomal instability (Castanogla et al (2005) 1756:115-125; Perera et al (2008) 29:747-53). Without wishing to be bound by theory, the present inventors predict that ras mutations may confer sensitivity to roscovitine treatment through reduced chromosomal stability. This indicates that roscovitine-based therapies may be effective for lung cancer patients resistant to epidermal growth factor receptor-tyrosine kinase inhibitor-based therapy due to activating ras mutations.

### Example 2

### Repeat Analysis Using Compound A to D

A repeat analysis was conducted with expansion to include the follow-on molecules Compounds A to D. There was extremely good correlation between the sensitivity of the cells to both roscovitine and Compound A (Pearson correlation=0.9) thereby implying that sensitivity to Compound A is also dependent on ras mutational status.

The cell line data are shown in Table 3 and 4.

### Example 3

### Investigating the Relationship Between Roscovitine Sensitivity and Sensitivity to Other Drugs

The cell line panel used in the Galimberti manuscript had previously been used to screen a number of other kinase inhibitors (McDermott 2007 PNAS vol 104(50): p 19936-41). When the data for the NSCLC cell lines that overlapped both studies was compared it was immediately apparent that the cell lines most sensitive to roscovitine were not the most sensitive to any of the other kinase inhibitors, in fact roscovitine had a unique profile. Moreover those cell lines more sensitive to roscovitine were less sensitive to EGFR inhibitors; this makes logical sense because EGFR inhibitors are known to be less active in cells that contain k-ras mutations

### Example 4

### Response of Patient with Mutant Ras Status to Roscovitine

The incidence of nasopharyngeal cancer (NPC) varies, with higher rates in southern Asia, intermediate rates in Mediterranean basin countries as well as in Greenland and Alaska populations, and low rates in most of the western countries (Chang et al 2006 Cancer Epidemiol. Biomarkers Prev. v15 p 1765). A clinical study of R-roscovitine (seliciclib) in patients with previously treated advanced solid tumors that was heavily enriched for NPC patients was performed. Patients were evaluated for 6-month progression-free survival and two dosing schedules of seliciclib were used. Schedule A was 400 mg seliciclib given twice per day for four consecutive days repeated every week. Schedule B was 800 mg seliciclib given once per day for four consecutive days repeated every week. Patients were also evaluated for overall survival, response rate, response duration, safety, and tolerability.

A total of 23 patients were enrolled on the study (11 at 400 mg bid and 12 at 800 mg qd.). Both dose levels were well tolerated and prolonged stable disease among the NPC patients was seen in a number of individuals (Yeo et al 2009 J. Clin. Oncol. 2009; 27:15s, (suppl; abstr 6026).

One NPC patient was treated on schedule B and experienced stable disease for over two years. This patient was Caucasian and thus not from one of the patient populations where NPC is more prevalent. K-Ras mutations are extremely rare in NPC patients. A biopsy sample from this patient was analysed for K-Ras mutational status. DNA was extracted from a paraffin embedded tumor sample and analysed for a total of 12 different mutations in Gly12 and Gly13 which represent the most common K-Ras activating mutations. Analysis was performed using a standard PCR-RFLP methodology as has been reported previously (Hatzaki et al 2001 Molecular and Cellular Probes; v15, 243) which identified a K-Ras activating mutation at Gly12 in the tumor sample.

Thus this patient represents an unusual individual to present with NPC, both being Caucasian and carrying an activating K-Ras mutation, however, he responded well to seliciclib achieving stable disease for greater than two years. This was a longer period of clinical benefit than achieved with four previous therapies.

### Example 5

### Clinical Study

Adult patients with histologically-confirmed recurrent non-small cell lung cancer were treated in a randomized Phase II study of R-roscovitine (seliciclib) oral capsules. Patients must have had at least two prior systemic treatment regimens and had measurable disease according to RECIST, with an Eastern Cooperative Oncology Group performance status 0-1, adequate bone marrow, hepatic and renal function and ability to swallow capsules. Patients were also at least 3 weeks from prior systemic treatments including investigational anti-cancer therapy, at least 7 days from prior radiation therapy and had recovered from prior toxicities. All patients were dosed with 1200 mg of seliciclib twice per day for three days every two weeks. This cycle of treatment was repeated three times. Patients achieving stable disease at this six week evaluation point were randomised to continue receiving seliciclib on the same schedule, or to receive placebo capsules on an identical schedule. A total of 187 patients were enrolled and treated. Fifty-three patients entered the randomised phase of treatment. Patients continued to be treated with seliciclib or placebo until they had progressive disease by RECIST criteria. Patients receiving placebo who then had progressive disease were permitted to cross over back onto the seliciclib treatment. Patients were evaluated for progression-free survival (PFS), PFS from the time of randomization, overall survival, response, response duration, safety and tolerability.

Where tumor biopsy samples were sufficient and available, an assessment of Ras status was made using a commercially available assay.

### Tables

TABLE 1
Fractional growth inhibitory response of human cancer cell lines following 72 hours
treatment with seliciclib (15 μM) displayed from most to least sensitive cell lines.

| | | | | Legend |
|---|---|---|---|---|
| | | | 15 μM | (Fractional |
| CellLine | Organ | Histology | seliciclib | Growth) |
| SNU-398 | Liver | hepatocellular | 0.0473 | >1.5 |
| | | carcinoma | | |
| PL4 | Pancreas | | 0.057 | .75-1.5 |
| LU99A | Lung: NSCLC | giant cell carcinoma | 0.0948 | .5-.75 |
| NCI-H2122 | Lung: NSCLC | non-small cell lung | 0.1093 | .2-.5 |
| | | cancer | | |
| SW 48 | Intestine | colon adenocarcinoma | 0.1123 | <.2 |
| T.T | Esophagus | squamous cell | 0.1271 | |
| | | carcinoma | | |
| EN | Uterus | endometrial carcinoma | 0.1503 | |
| COLO 853 | Skin | malignant melanoma | 0.154 | |
| HLE | Liver | hepatoma | 0.1553 | |
| HMCB | Skin | melanoma | 0.161 | |
| LCLC-97TM1 | Lung: NSCLC | large cell lung | 0.1711 | |
| | | carcinoma | | |
| A-375 | Skin | melanoma | 0.1771 | |
| Detroit 562 | Head & Neck | pharynx carcinoma | 0.1846 | |
| A549 | Lung: NSCLC | carcinoma | 0.1925 | |
| RPMI 2650 | Head & Neck | nasal septum quasi- | 0.1937 | |
| | | diploid squamous | | |
| | | carcinoma | | |
| HT 1080 | Miscellaneous | fibrosarcoma | 0.2044 | |
| AN3CA | Uterus | endometrial | 0.2094 | |
| | | adenocarcinoma | | |
| AGS | Stomach | gastric adenocarcinoma | 0.2157 | |
| LU99B | Lung: NSCLC | giant cell carcinoma | 0.2318 | |
| COR-L23 | Lung: NSCLC | large cell carcenoma | 0.2341 | |
| RT-112 | Bladder | urinary bladder | 0.243 | |
| | | transitional cell | | |
| PFSK-1 | Brain | cerebellum | 0.249 | |
| HCC-366 | Lung: NSCLC | non-small cell lung | 0.2561 | |
| | | carcinoma | | |
| PCI-15 | Head & Neck | | 0.2641 | |
| A-204 | Muscle | rhabdomyosarcoma | 0.2653 | |
| JHU-013 | Head & Neck | | 0.2699 | |
| G-402 | Kidney | renal leiomyoblastoma | 0.2708 | |
| HGC-27 | Stomach | gastric carcinoma | 0.2806 | |
| Lu-135 | Lung | small cell lung | 0.2812 | |
| | | carcinoma | | |
| SK-N-MC | Brain | neuroepithelioma | 0.283 | |
| PCI-15B | Head & Neck | | 0.2847 | |
| NCI-H1734 | Lung: NSCLC | non-small cell lung | 0.2849 | |
| | | cancer | | |
| CS1R | Bone | | 0.2923 | |
| TYK-nu | Ovary | carcinoma | 0.3005 | |
| KU-19-19 | Bladder | urinary bladder | 0.3033 | |
| | | transitional cell | | |
| NCI-H2030 | Lung: NSCLC | non-small cell lung | 0.3063 | |
| | | cancer | | |
| EJ138 | Bladder | bladder carcinoma | 0.3103 | |
| A431 | Skin | squamous carcinoma | 0.3116 | |
| CAL-62 | Thyroid | thyroid anaplastic | 0.3139 | |
| | | carcinoma | | |
| UO-31 | Kidney | | 0.3152 | |
| TOV-112D | Ovary | endometrioid | 0.3156 | |
| | | carcinoma | | |
| RT112/84 | Bladder | bladder carcinoma | 0.318 | |
| | | epithelial | | |
| COLO 320DM | Intestine | carcinoma of sigmoid | 0.3188 | |
| | | colon | | |
| SAS | Head & Neck | tongue squamous | 0.3207 | |
| | | carcinoma | | |
| JHH-6 | Liver | undifferentiated | 0.3234 | |
| | | hepatocellular | | |
| | | carcinoma | | |
| NCI-H2347 | Lung: NSCLC | non-small cell lung | 0.3299 | |
| | | cancer | | |
| VM-CUB1 | Bladder | urinary bladder | 0.3307 | |
| | | transitional cell | | |
| NCI-H1568 | Lung: NSCLC | non-small cell lung | 0.3317 | |
| | | cancer | | |
| NCI-H1299 | Lung: NSCLC | non-small cell lung | 0.3352 | |
| | | cancer | | |
| SNG-M | Uterus | adenocarcinoma | 0.3393 | |
| KP-3 | Pancreas | adenosquamous | 0.3396 | |
| | | carcinoma | | |
| NAE | Skin | melanoma | 0.3413 | |
| XPA-4 | Pancreas | | 0.3444 | |
| CAL-12T | Lung: NSCLC | non-small cell lung | 0.3472 | |
| | | carcinoma | | |
| UM-UC-3 | Bladder | transitional cell | 0.3538 | |
| | | carcinoma | | |
| G-401 | Kidney | rhabdoid tumor | 0.3542 | |
| | | (formerly classified as | | |
| | | Wilms' tumor | | |
| MS751 | Cervix | cervical carcinoma | 0.3573 | |
| MKN1 | Stomach | adenosquamous | 0.3594 | |
| PCI-4A | Head & Neck | squamous cell | 0.3628 | |
| | | carcinoma | | |
| PCI-38 | Head & Neck | H&N | 0.3656 | |
| LS174T | Intestine | colon adenocarcinoma | 0.3713 | |
| JHU-028EP | Head & Neck | | 0.3716 | |
| SW 780 | Bladder | transitional cell | 0.3743 | |
| | | carcinoma | | |
| M-14 | Skin | | 0.3765 | |
| LU65C | Lung: NSCLC | giant cell carcinoma | 0.3767 | |
| LU99C | Lung: NSCLC | giant cell carcinoma | 0.3864 | |
| Caki-1 | Kidney | Renal | 0.3885 | |
| SU.86.86 | Pancreas | adenocarcinoma | 0.3887 | |
| H2052 | Lung | pleural mesothelioma | 0.3892 | |
| HSC-4 | Head & Neck | tongue squamous | 0.3894 | |
| | | carcinoma | | |
| HuH-7 | Liver | hepatoma | 0.3911 | |
| HTC-C3 | Thyroid | throid carcinoma | 0.3914 | |
| MES-SA | Uterus | uterus sarcoma | 0.3922 | |
| FTC-133 | Thyroid | follicular thyroid | 0.393 | |
| | | carcinoma | | |
| EBC-1 | Lung: NSCLC | squamous cell | 0.3971 | |
| | | carcinoma | | |
| 786-O | Kidney | renal cell | 0.3998 | |
| | | adenocarcinoma | | |
| PC-3 | Prostate | adenocarcinoma | 0.4021 | |
| OVCAR-5 | Ovary | | 0.4053 | |
| KYSE-450 | Esophagus | esophageal squamous | 0.4067 | |
| | | cell carcinoma | | |
| OE21 | Esophagus | esophageal squamous | 0.4083 | |
| | | cell carcinoma | | |
| Hs 633T | Miscellaneous | fibrosarcoma | 0.41 | |
| A673 | Muscle | rhabdomyosarcoma | 0.4111 | |
| NCI-H1975 | Lung: NSCLC | adenocarcinoma; non- | 0.4117 | |
| | | small cell lung cancer | | |
| MCF7 | Breast | adenocarcinoma | 0.4137 | |
| KYSE-510 | Esophagus | esophageal squamous | 0.4141 | |
| | | cell carcinoma | | |
| NCI-H2023 | Lung: NSCLC | non-small cell lung | 0.4148 | |
| | | cancer | | |
| BEN | Lung: NSCLC | lung carcinoma | 0.4149 | |
| RVH-421 | Skin | melanoma | 0.4165 | |
| 639-V | Bladder | ureter transitional cell | 0.4196 | |
| | | carcinoma | | |
| BFTC-905 | Bladder | urinary bladder | 0.4198 | |
| | | transitional cell | | |
| 8505C | Thyroid | thyroid carcinoma | 0.4214 | |
| Panc 08.13 | Pancreas | adenocarcinoma | 0.4222 | |
| JHU-019 | Head & Neck | | 0.4229 | |
| A-427 | Lung: NSCLC | carcinoma | 0.4252 | |
| Hs 766T | Pancreas | adenocarcinoma | 0.4284 | |
| KATO III | Stomach | signet ring | 0.4299 | |
| | | adenocarcinoma | | |
| SiHa | Cervix | cervical squamous cell | 0.4302 | |
| | | carcinoma | | |
| HUP-T3 | Pancreas | Carcinoma | 0.4333 | |
| NCI-H1915 | Lung: NSCLC | non-small cell lung | 0.4337 | |
| | | cancer | | |
| KP-3L | Pancreas | adenosquamous | 0.4338 | |
| | | carcinoma | | |
| ESS-1 | Uterus | endometrial stromal | 0.4399 | |
| | | sarcoma | | |
| T98G | Brain | glioblastoma | 0.4404 | |
| COLO 205 | Intestine | colon adenocarcinoma | 0.4437 | |
| SK-MES | Lung: NSCLC | squamous carcinoma | 0.4439 | |
| BHT-101 | Thyroid | thyroid carcinoma | 0.4486 | |
| NCI-H647 | Lung: NSCLC | non-small cell lung | 0.4521 | |
| | | cancer | | |
| NCI-H2170 | Lung: NSCLC | squamous cell | 0.4534 | |
| | | carcinoma | | |
| MGH-MC-1 | Skin | melanoma | 0.4543 | |
| HuCCT1 | Liver | bile duct carcinoma | 0.4559 | |
| NCI-H2452 | Lung | mesothelioma | 0.4571 | |
| NCI-H2085 | Lung: NSCLC | non-small cell lung | 0.4597 | |
| | | cancer | | |
| Ca9-22 | Head & Neck | gingival carcinoma | 0.4623 | |
| LU65A | Lung: NSCLC | giant cell carcinoma | 0.4627 | |
| HPAF-II | Pancreas | adenocarcinoma | 0.4628 | |
| HEC-1 | Uterus | endometrial | 0.4632 | |
| | | adenocarcinoma | | |
| MDA-MB-231 | Breast | adenocarcinoma | 0.4643 | |
| HMVII | Skin | vaginal malignant | 0.4646 | |
| | | melanoma | | |
| NCI-H1792 | Lung: NSCLC | adenocarcinoma | 0.4649 | |
| SW 156 | Kidney | hypernephroma | 0.467 | |
| PCI-4B | Head & Neck | | 0.4686 | |
| KYSE-410 | Esophagus | esophageal squamous | 0.4693 | |
| | | cell carcinoma | | |
| J82 | Bladder | transitional cell | 0.4753 | |
| | | carcinoma | | |
| AU565 | Breast | adenocarcinoma | 0.4762 | |
| NCI-H1703 | Lung: NSCLC | non-small cell lung | 0.4772 | |
| | | cancer | | |
| IGROV-1 | Ovary | | 0.4776 | |
| GCT | Miscellaneous | fibrous histiocytoma | 0.4799 | |
| HCC-827 | Lung: NSCLC | non-small cell lung | 0.4812 | |
| | | carcinoma | | |
| MIA PaCa-2 | Pancreas | adenocarcinoma | 0.4826 | |
| A2780 | Ovary | ovarian carcinoma | 0.4827 | |
| HT115 | Intestine | colon carcinoma | 0.4837 | |
| HO-1-u-1 | Head & Neck | mouth floor squamous | 0.4858 | |
| | | cell carcinoma | | |
| Panc 04.03 | Pancreas | adenocarcinoma | 0.4899 | |
| MDA-H2774 | Ovary | | 0.491 | |
| UMC-11 | Lung | carcinoma | 0.4912 | |
| Panc 10.05 | Pancreas | adenocarcinoma | 0.493 | |
| MEL-JUSO | Skin | melanoma | 0.4945 | |
| PCI-15A | Head & Neck | | 0.4965 | |
| WM 266-4 | Skin | melanoma | 0.4972 | |
| ACHN | Kidney | renal cell | 0.4977 | |
| | | adenocarcinoma | | |
| HT-1197 | Bladder | carcinoma | 0.5024 | |
| LCLC-103H | Lung: NSCLC | large cell lung | 0.5051 | |
| | | carcinoma | | |
| C-4 I | Cervix | cervical carcinoma | 0.5067 | |
| WM-115 | Skin | melanoma | 0.5068 | |
| H2869 | Lung | pleural mesothelioma | 0.5086 | |
| AZ-521 | Stomach | adenocarcinoma | 0.5115 | |
| SK-MEL-37 | Skin | melanoma | 0.5209 | |
| NH-6 | Kidney | adrenal gland | 0.5218 | |
| | | neuroblastoma | | |
| HSC-3 | Head & Neck | tongue squamous | 0.5223 | |
| | | carcinoma | | |
| HT-29 | Intestine | colorectal | 0.5243 | |
| | | adenocarcinoma | | |
| T24 | Bladder | transitional cell | 0.5262 | |
| | | carcinoma | | |
| JHU-028 | Head & Neck | | 0.5325 | |
| PCI-6B | Head & Neck | | 0.5328 | |
| HCT-15 | Intestine | colon adenocarcinoma | 0.5363 | |
| T84 | Intestine | colon carcinoma | 0.5371 | |
| C32 | Skin | melanoma | 0.5391 | |
| HSC-2 | Head & Neck | mouth squamous | 0.5464 | |
| | | carcinoma | | |
| MG-63 | Bone | osteosarcoma | 0.5484 | |
| NCI-H1573 | Lung: NSCLC | adenocarcinoma | 0.5485 | |
| NCI-H1048 | Lung | small cell lung cancer | 0.5502 | |
| KMRC-1 | Kidney | carcinoma | 0.554 | |
| PCI-30 | Head & Neck | | 0.5541 | |
| NUGC-3 | Stomach | carcinoma | 0.5557 | |
| PA-TU-8902 | Pancreas | adenocarcinoma | 0.5577 | |
| BHY | Head & Neck | oral squamous cell | 0.5602 | |
| | | carcinoma | | |
| KP-4 | Pancreas | ductal cell carcinoma | 0.5607 | |
| SW 13 | Kidney | adrenal cortex | 0.5624 | |
| | | adenocarcinoma | | |
| Panc 03.27 | Pancreas | adenocarcinoma | 0.5632 | |
| SCaBER | Bladder | squamous cell | 0.5642 | |
| | | carcinoma | | |
| C-33 A | Cervix | carcinoma | 0.5677 | |
| MFM-223 | Breast | breast carcinoma | 0.5683 | |
| U-2 OS | Bone | osteosarcoma | 0.5686 | |
| GAMG | Brain | glioma | 0.571 | |
| SCC-25 | Head & Neck | tongue squamous cell | 0.571 | |
| | | carcinoma | | |
| Saos-2 | Bone | osteosarcoma | 0.5733 | |
| CAL-29 | Bladder | urinary bladder | 0.5744 | |
| | | transitional cell | | |
| B-CPAP | Thyroid | thyroid carcinoma | 0.5754 | |
| PANC-1 | Pancreas | ductal adenocarcinoma | 0.5761 | |
| IGR-1 | Skin | melanoma | 0.5764 | |
| PC-14 | Lung: NSCLC | adenocarcinoma | 0.5776 | |
| PA-TU-8988S | Pancreas | Adenocarcinoma | 0.5791 | |
| CaR-1 | Intestine | rectum adenocarcinoma | 0.5795 | |
| PCI-6A | Head & Neck | | 0.5818 | |
| SW 900 | Lung: NSCLC | non-small cell lung | 0.5842 | |
| | | cancer | | |
| NCI-H727 | Lung | carcinoma bronchus | 0.5866 | |
| YAPC | Pancreas | Carcinoma | 0.588 | |
| OVCAR-8 | Ovary | | 0.5886 | |
| Ca Ski | Cervix | cervical epidermoid | 0.591 | |
| | | carcinoma | | |
| MFE-280 | Uterus | endometrial | 0.5959 | |
| | | adenocarcinoma | | |
| VMRC-RCZ | Kidney | renal cancer | 0.5994 | |
| 769-P | Kidney | renal cell | 0.602 | |
| | | adenocarcinoma | | |
| BPH-1 | Prostate | benign prostate | 0.6039 | |
| | | hyperplasia | | |
| KP-1N | Pancreas | pancreatic tumor | 0.6045 | |
| SW 1573 | Lung: NSCLC | alveolar cell carcinoma | 0.6076 | |
| Calu-1 | Lung: NSCLC | non-small cell lung | 0.6124 | |
| | | cancer | | |
| 143B | Bone | osteosarcoma | 0.6124 | |
| 5637 | Bladder | carcinoma | 0.6151 | |
| H2373 | Lung | pleural mesothelioma | 0.6168 | |
| HOS | Bone | osteosarcoma | 0.617 | |
| SHP-77 | Lung | | 0.617 | |
| SCCH-196 | Miscellaneous | small round cell | 0.6177 | |
| | | sarcoma | | |
| HARA | Lung: NSCLC | squamous cell lung | 0.6182 | |
| | | carcinoma | | |
| NCI-H2172 | Lung: NSCLC | non-small cell lung | 0.6197 | |
| | | cancer | | |
| DoTc2 4510 | Cervix | cervical carcinoma | 0.6205 | |
| Daoy | Brain | medulloblastoma | 0.6209 | |
| NCI-H522 | Lung: NSCLC | non-small cell lung | 0.6262 | |
| | | cancer | | |
| NCI-H1781 | Lung: NSCLC | adenocarcinoma | 0.6273 | |
| NCI-H1793 | Lung: NSCLC | adenocarcinoma, non- | 0.6274 | |
| | | small cell lung cancer | | |
| KYSE-140 | Esophagus | esophageal squamous | 0.6278 | |
| | | cell carcinoma | | |
| NCI-H1651 | Lung: NSCLC | non-small cell lung | 0.6289 | |
| | | cancer | | |
| JHU-022 | Head & Neck | | 0.6294 | |
| EPLC-272H | Lung: NSCLC | epidermoid lung | 0.6305 | |
| | | carcinoma | | |
| SNU-449 | Liver | hepatocellular | 0.6309 | |
| | | carcinoma | | |
| T47D | Breast | breast tumor | 0.6314 | |
| A2058 | Skin | melanoma | 0.6341 | |
| JHH-2 | Liver | hepatocellular | 0.635 | |
| | | carcinoma | | |
| NCI-H1435 | Lung: NSCLC | non-small cell lung | 0.6379 | |
| | | cancer | | |
| SN-12C | Kidney | renal cell carcinoma | 0.6383 | |
| G-292 Clone | Bone | osteosarcoma | 0.6393 | |
| A141B1 | | | | |
| TCCSUP | Bladder | transitional cell | 0.6407 | |
| | | carcinoma | | |
| RERF-GC-1B | Stomach | adenocarcinoma | 0.6411 | |
| SISO | Cervix | cervix adenocarcinoma | 0.6424 | |
| Sarc9371 | Bone | sarcoma | 0.6425 | |
| RERF-LC-Sq1 | Lung: NSCLC | squamous carcinoma | 0.6428 | |
| Calu-3 | Lung: NSCLC | adenocarcinoma | 0.6463 | |
| 1A6 | Bladder | carcinoma | 0.6475 | |
| GTL-16 | Stomach | | 0.6482 | |
| SW756 | Cervix | cervical squamous cell | 0.6484 | |
| | | carcinoma | | |
| NCI-H2228 | Lung: NSCLC | non-small cell lung | 0.6493 | |
| | | cancer | | |
| ABC-1 | Lung: NSCLC | adenocarcinoma | 0.6543 | |
| SKG-IIIb | Cervix | squamous cell | 0.6636 | |
| | | carcinoma | | |
| RCM-1 | Intestine | rectum adenocarcinoma | 0.6689 | |
| BxPC-3 | Pancreas | adenocarcinoma | 0.671 | |
| CHSA8926 | Bone | chondrosarcoma | 0.674 | |
| SW-1710 | Bladder | urinary bladder | 0.6764 | |
| | | transitional cell | | |
| CAL-33 | Head & Neck | tongue squamous cell | 0.6834 | |
| | | carcinoma | | |
| COLO-680N | Esophagus | esophagus squamous | 0.6838 | |
| | | cell carcinoma | | |
| DAN-G | Pancreas | Adenocarcinoma | 0.6841 | |
| NCI-H520 | Lung: NSCLC | squamous cell | 0.6863 | |
| | | carcinoma | | |
| AsPC-1 | Pancreas | adenocarcinoma | 0.6879 | |
| HT 1376 | Bladder | bladder carcinoma | 0.6897 | |
| NCI-H1944 | Lung: NSCLC | non-small cell lung | 0.6921 | |
| | | cancer | | |
| LNZTA3WT4 | Brain | glioblastoma | 0.6936 | |
| S-117 | Thyroid | thyroid sarcoma | 0.696 | |
| HCC1395 | Breast | primary ductal | 0.6999 | |
| | | carcinoma | | |
| BT-20 | Breast | carcinoma | 0.7016 | |
| LUDLU-1 | Lung: NSCLC | squamous cell | 0.7029 | |
| | | carcinoma | | |
| HPAC | Pancreas | adenocarcinoma | 0.7062 | |
| BFTC-909 | Kidney | kidney transitional cell | 0.7132 | |
| | | carcinoma | | |
| DBTRG-05MG | Brain | glioblastoma | 0.7139 | |
| SK-N-AS | Brain | neuroblastoma | 0.7261 | |
| SK-OV-3 | Ovary | ovary adenocarcinoma | 0.7375 | |
| GP5d | Intestine | colon adenocarcinoma | 0.7382 | |
| SBC-5 | Lung | small cell carcinoma | 0.7399 | |
| 22RV1 | Prostate | prostate carcinoma | 0.7432 | |
| SW620 | Intestine | | 0.7486 | |
| H2596 | Lung | pleural mesothelioma | 0.7486 | |
| C-4 II | Cervix | cervical carcinoma | 0.7489 | |
| NCI-H630 | Intestine | colorectal | 0.7543 | |
| | | adenocarcinoma | | |
| BT-549 | Breast | ductal carcinoma | 0.758 | |
| SW 1116 | Intestine | colon adenocarcinoma | 0.7592 | |
| NCI-H841 | Lung | small cell lung cancer | 0.7639 | |
| NCI-H1581 | Lung: NSCLC | lung cancer | 0.7659 | |
| PC-9 | Lung: NSCLC | non-small cell lung | 0.7822 | |
| | | cancer | | |
| HDQ-P1 | Breast | breast carcinoma | 0.8137 | |
| HCC38 | Breast | primary ductal | 0.8297 | |
| | | carcinoma | | |
| DK-MG | Brain | glioma | 0.8572 | |
| H2722 | Lung | pleural mesothelioma | 0.8974 | |
| HeLa | Cervix | cervical | 0.9192 | |
| | | adenocarcinoma | | |
| RT4 | Bladder | bladder transitional cell | 0.9342 | |
| | | carcinoma | | |
| H4 | Brain | glioma | 0.9708 | |

TABLE 2A
Ras status for the 15 NSCLC cell lines with
highest growth inhibitory response to seliciclib.
Most Seliciclib Sensitive NSCLC Cells

| | | Fractional Growth |
|---|---|---|
| Cell Line | RAS Status | Seliciclib |
| LU99A | Mutant (K-RAS) | 0.0948 |
| NCI-H2122 | Mutant (K-RAS) | 0.1093 |
| LCLC-97TM1 | Mutant (K-RAS) | 0.1711 |
| A549 | Mutant (K-RAS) | 0.1925 |
| LU99B | Mutant (K-RAS) | 0.2318 |
| COR-L23 | Mutant (K-RAS) | 0.2341 |
| HCC-366 | Not Known | 0.2561 |
| NCI-H1734 | Mutant (K-RAS) | 0.2849 |
| NCI-H2030 | Mutant (K-RAS) | 0.3063 |
| NCI-H2347 | Mutant (K-RAS) | 0.3299 |
| NCI-H1568 | Not Known | 0.3317 |
| NCI-H1299 | Mutant (N-RAS) | 0.3352 |
| CAL-12T | Wild-type | 0.3472 |
| LU65C | Mutant (K-RAS) | 0.3767 |
| LU99C | Mutant (K-RAS) | 0.3864 |

TABLE 2B
Ras status for the 15 NSCLC cell lines with
least growth inhibitory response to seliciclib.
Least Seliciclib Sensitive NSCLC Cells

| | | Fractional Growth |
|---|---|---|
| Cell Line | RAS Status | Seliciclib |
| NCI-H522 | Wild-type | 0.6262 |
| NCI-H1781 | Wild-type | 0.6273 |
| NCI-H1793 | Wild-type | 0.6274 |
| NCI-H1651 | Wild-type | 0.6289 |
| EPLC-272H | Wild-type | 0.6305 |
| NCI-H1435 | Wild-type | 0.6379 |
| RERF-LC-Sq1 | Wild-type | 0.6428 |
| Calu-3 | Wild-type | 0.6463 |
| NCI-H2228 | Wild-type | 0.6493 |
| ABC-1 | Wild-type | 0.6543 |
| NCI-H520 | Wild-type | 0.6863 |
| NCI-H1944 | Wild-type | 0.6921 |
| LUDLU-1 | Wild-type | 0.7029 |
| NCI-H1581 | Wild-type | 0.7659 |
| PC-9 | Wild-type | 0.7822 |

TABLE 3
Cell line data for compounds A, B, C and D and roscovitine

| | | | | IC50 micromolar | | | | |
|---|---|---|---|---|---|---|---|---|
| | Ras | | | R- | | | | |
| Cell Line | Status | Mutation | Tissue | roscovitine | B | C | D | A |
| H1650 | WT | | Lung | 21.9 | 6.5 | 3.7 | 1.2 | 0.5 |
| MDA-MB-436 | WT | | Breast | 17.8 | 6.6 | 3.4 | 1.0 | 0.4 |
| H2052 | WT | | Mesothelioma | 17.1 | 5.2 | 2.4 | 0.9 | 0.3 |
| LoVo | K-Ras | G13D | Colon | 17.1 | 4.5 | 2.2 | 0.8 | 0.7 |
| Saos-2 | WT | | Osteosarcoma | 17.0 | 5.3 | 2.5 | 1.4 | |
| H292 | WT | | Lung | 15.4 | 6.6 | 2.3 | 0.9 | 0.4 |
| Colo205 | WT | | Colon | 13.3 | 4.5 | 2.3 | 0.8 | 0.3 |
| HT-29 | WT | | Colon | 12.5 | 4.1 | 1.7 | 1.2 | |
| NCI-H460 | K-Ras | Q61H | Lung | 12.3 | 2.8 | 2.2 | 0.7 | 0.5 |
| LP-1 | WT | | Myeloma | 11.7 | | 1.6 | 0.5 | |
| A549 | K-Ras | G12S | Lung uterine | 10.5 | 3.0 | 1.6 | 0.5 | 0.2 |
| MESSA | WT | | sarcoma | 10.0 | 3.6 | 1.6 | 0.5 | 0.2 |
| HCT 116 | K-Ras | G13D | Colon | 9.5 | | 1.6 | 0.4 | |
| MCF7 | WT | | Breast | 9.4 | 3.6 | 1.6 | 0.4 | 0.3 |
| NCI-H929 | N-Ras | | Myeloma | 8.1 | 5.2 | 2.4 | 0.8 | 0.4 |
| A2780 | WT | | Ovary | 6.7 | 2.6 | 1.1 | 0.4 | 0.4 |
| H358 | K-Ras | G12C | Lung | 5.8 | 2.7 | 0.8 | 0.3 | 0.2 |
| Median | | | | 12.3 | 4.5 | 2.2 | 0.8 | 0.4 |
| IC50 | | | | | | | | |

TABLE 4

| | Number of cell lines with |
|---|---|
| | mutant Ras status with IC50 |
| Compound | values below the median IC50 |
| R-roscovitine | 5 out of 6 |
| Compound B | 4 out of 5 |
| Compound C | 5 out of 6 |
| Compound D | 6 out of 6 |
| Compound A | 3 out of 5 |

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in cancer biology or related fields are intended to be within the scope of the following claims.

1. A method for determining whether or not a cancer subject is suitable for treatment with a purine-based roscovitine-like inhibitor, which method comprises the step of determining the ras status of the cancer,
wherein a determination that the subject has mutant ras status is indicative that the subject is suitable for treatment with a purine-based roscovitine-like inhibitor.

2. A method according to claim 1, wherein a determination that the subject has wild-type ras status is indicative that the subject is unsuitable for treatment with a purine-based roscovitine-like inhibitor.

3. A method for selecting a therapy for treating a cancer subject which comprises the step of determining whether the subject is suitable for treatment with a purine-based roscovitine-like inhibitor using a method according to claim 1, and selecting treatment with a purine-based roscovitine-like inhibitor if the subject has mutant ras status.

4. A method for selecting a therapy for treating a cancer subject which comprises the step of determining whether the subject is suitable for treatment with a purine-based roscovitine-like inhibitor using a method according to claim 2, and selecting an alternative type of treatment if the subject has wild-type ras status.

5. A method for treating cancer in a subject by administering a therapeutically effective amount of a purine-based roscovitine-like inhibitor to the subject, wherein the subject has a cancer characterised by mutant ras status.

6. A method for treating cancer in a subject, which comprises the following steps:
(i) determining the ras status of the cancer; and
(ii) administering a therapeutically effective amount of a purine-based roscovitine-like inhibitor to the subject, if the cancer has mutant ras status.

7. A method according to claim 3, wherein the treatment involves the use of a purine-based roscovitine-like inhibitor in combination with another therapeutic agent.

8. A method according to claim 7, wherein the treatment involves the use of a purine-based roscovitine-like inhibitor in combination with a receptor tyrosine kinase (RTK) inhibitor.

9. A method according to claim 8, wherein the treatment involves the use of a purine-based roscovitine-like inhibitor in combination with an EGF-R inhibitor and/or a MEK inhibitor.

10. A method according to claim 7, wherein the treatment involves the use of a purine-based roscovitine-like inhibitor in combination with an m-TOR inhibitor.

11. A method according to claim 7, wherein the treatment involves the use of a purine-based roscovitine-like inhibitor in combination with a PI3-kinase inhibitor.

12. A method according to claim 3, wherein the treatment involves the use of a purine-based roscovitine-like inhibitor in combination with a prodrug or pharmaceutical preparation in which the active ingredient is a microtubule targeting agent.

13. A method according to claim 12, wherein the microtubule targeting agent is paclitaxel, docetaxel or a taxane.

14. A method according to claim 3, wherein the purine-based roscovitine-like inhibitor is selected from roscovitine, Compound A, B, C and D, bohemine and olomoucine.

15. A method according to claim 14, wherein the purine-based roscovitine-like inhibitor is roscovitine.

16. A method according to claim 3, wherein a subject having mutant ras status expresses K-ras, H-ras or N-ras mutant protein.

17. A method according to claim 1, wherein the cancer is selected from lung, pancreas, colorectal, breast, liver, intestine, oesophagus, uterus, skin, head & neck, nasopharyngeal and haematological cancer, such as Acute Myeloid Leukemia (AML).

18. A method according to claim 17, wherein the cancer is lung or colorectal cancer.

19. A method according to claim 18, wherein the cancer is non small-cell lung carcinoma (NSCLC).

20. A method according to claim 1, wherein the cancer is insensitive to chemotherapy with other agents.

21. A method according to claim 20, wherein the cancer is insensitive to chemotherapy with cytotoxic agents.

22. A method according to claim 21, wherein the cancer is insensitive to treatment with targeted agents such as EGFR inhibitors and mTOR inhibitors.
